(19) United States
(12) Patent Application Publication
(10) Pub. No.: US 2003/0152534 A1
(43) Pub. Date: Aug. 14, 2003

(54) Composition for dyeing keratin fibers comprising a particular aminosilicone

(76) Inventors: Frederic Legrand, Courbevoie (FR); Jean-Marie Millequant, Saint-Maur des Fosses (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/290,345

(22) Filed: Nov. 8, 2002

(30) Foreign Application Priority Data
Nov. 8, 2001 (FR) 01 14469

(51) Int. Cl. A61K 7/04

(52) U.S. Cl. 424/61

(57) ABSTRACT

Composition for dyeing keratin fibers, such as human hair, comprising, in a cosmetically acceptable medium, at least one coloring agent chosen from direct dyes and oxidation dyes and an aminoethylimino(C₄-C₈)alkyl amino silicone. Dyeing devices, kits and methods using said compositions are also disclosed.

This disclosure relates to a composition for dyeing keratin fibers such as human hair, comprising at least one coloring agent chosen from direct dyes and oxidation dyes and at least one aminosilicone comprising at least one aminoethylimino(C₄-C₈)alkyl group.

It is known that there are two types of dyeing for keratin fibers: direct dyeing, employing direct dyes and/or pigments which are colored molecules and which give the fibers a temporary color which may fade after several shampooings, and which is carried out in the presence or absence of oxidizing agents; and so-called oxidation dyeing, which employs oxidation dye precursors and an oxidizing agent and which may impart to the fibers a color which is more long-lasting than that of direct dyeing.

It is also known that the use of an oxidizing agent involves a certain degree of degradation of the keratin fiber.

At the present time a very marked trend is being observed towards an increase in the frequency of shampooing, one outcome of which is greater degradation of the dyeings between two applications.

Consequently there exists a need to improve the durability of direct or oxidation dyeings, for example, with respect to shampoos.

The inventors have discovered, quite unexpectedly and surprisingly, that the use of a composition comprising at least one coloring agent chosen from direct dyes and oxidation dyes and at least one aminosilicone comprising at least one aminoethylimino(C₄-C₈)alkyl group may allow this problem to be solved.

Furthermore, it may allow the condition of the fibers to be improved.

As used herein, the phrase “improvement in the condition of the fiber”means a reduction in the porosity or the alkaline solubility of the fiber and an improvement in at least one cosmetic property, for example, in the smoothness, softness and ease of disentangling and of styling.

This effect can be remanent, ie., long-lasting.

The porosity is measured by fixing at 37° C. and a pH of 10, for 2 minutes, 2-nitro-para-phenylenediamine at 0.25% in an ethanol/pH 10 buffer mixture ( 10/90 volume ratio).

The alkaline solubility corresponds to the loss in mass of a sample of 100 mg of keratin fibers under the action of decinormal sodium hydroxide solution at 65° C. for 30 minutes. This discovery forms at least one portion of the basis for at least one embodiment disclosed herein.

One new embodiment therefore relates to a composition for dyeing human keratin fibers, such as hair, comprising, in a cosmetically acceptable medium, at least one coloring agent chosen from direct dyes and oxidation dyes, and at least one aminosilicone comprising at least one aminoethylimino(C₄-C₈)alkyl group.

Another new embodiment comprises a ready-to-use composition for dyeing keratin fibers which comprises at least one coloring agent chosen from direct dyes and oxidation dyes and at least one aminosilicone comprising at least one aminoethylimino(C₄-C₈)alkyl group, and an oxidizing agent.

As used herein, a “ready-to-use composition” is the composition intended for application as it is to the keratin fibers; that is, it may be stored as it is before use or may result from the extemporaneous mixing of two or more compositions.

Another new embodiment relates to a method of dyeing human keratin fibers, such as hair, which comprises applying to the fibers at least one coloring composition comprising, in a cosmetically acceptable medium, at least one direct dye and at least one aminosilicone comprising at least one aminoethylimino(C₄-C₈)alkyl group.

Further, another new embodiment relates to a method of dyeing human keratin fibers, such as hair, which comprises applying to the fibers at least one coloring composition comprising, in a cosmetically acceptable medium, at least one coloring agent chosen from direct dyes and oxidation dyes and at least one aminosilicone comprising at least one aminoethylimino(C₄-C₈)alkyl group, the color being developed at alkaline, neutral or acidic pH by means of a composition comprising at least one oxidizing agent, which is mixed at the time of use with the coloring composition or applied sequentially without rinsing in between.

Another new embodiment further relates to multi-compartment dyeing devices or kits.

A multi-compartment device or kit according to one new embodiment comprises at least two compartments wherein a first compartment comprises a composition comprising, in a cosmetically acceptable medium, at least one coloring agent chosen from direct dyes and oxidation dyes and at least one aminosilicone comprising at least one aminoethylimino(C₄-C₈)alkyl group and a second compartment comprises a composition comprising, in a cosmetically acceptable medium, at least one oxidizing agent.

Another new embodiment relates to a multi-compartment kit or device comprising at least three compartments, wherein a first compartment comprises a composition comprising, in a cosmetically acceptable medium, at least one coloring agent chosen from direct dyes and oxidation dyes, a second compartment comprises a composition comprising, in a cosmetically acceptable medium, at least one oxidizing agent, and a third compartment comprises a composition comprising, in a cosmetically acceptable medium, at least one aminosilicone comprising at least one aminoethylimino(C₄-C₈)alkyl group.

Additional characteristics, aspects, subject matter and advantages of the embodiments disclosed herein will appear even more clearly on reading the description and examples which follow without, however, exhibiting a limiting character.

Aminosilicones

An illustrative at least one aminosilicone has the following formula:

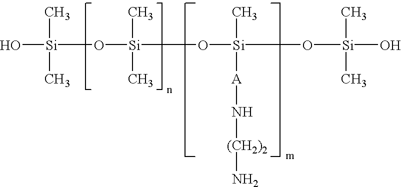

wherein:

A is chosen from linear and branched C₄-C₈ alkylene radicals, for example, C₄ alkylene radicals and

m and n are numbers such that the sum (n+m) can range, for example, from 1 to 2000 and, further for example, from 50 to 150,

n can be a number ranging from 0 to 1999, for example, from 49 to 149, and

m can be a number ranging from 1 to 2000, for example, from 1 to 10.

The term “alkylene radical” means divalent saturated hydrocarbon-based groups.

The viscosity of the at least one aminosilicone, for example, can be greater than 25,000 mm²/s at 25° C.

For example, this viscosity can range from 30,000 to 200,000 mm²/s at 25° C. and further, for example, from 30,000 to 150,000 mm²/s at 25° C.

The viscosity of the at least one aminosilicone is measured at 25° C. according to the standard “ASTM 445 Appendix C”.

The at least one aminosilicone has a weight-average molecular mass ranging, for example, from 2000 to 1,000,000 and further for example, from 3500 to 200,000.

The weight-average molecular mass of the at least one aminosilicone is measured by gel permeation chromatography (GPC) at ambient temperature, as polystyrene equivalents. The columns used are μ Styragel columns. The eluent is THF and the flow rate is 1 ml/min. 200 μl of a 0.5% by weight solution of silicone in THF are injected. The detection is carried out by refractometry and UVmetry.

When the at least one aminosilicone is used, one new embodiment involves using the at least one aminosilicone in the form of an oil-in-water emulsion. The oil-in-water emulsion may comprise at least one surfactant. The at least one surfactant may be of any nature, for example, cationic and/or nonionic.

The particles of silicone in the emulsion may have an average size ranging, for example, from 3 to 500 nanometers, further, for example, from 5 to 300 nanometers, even further, for example, from 10 to 275 nanometers and still further, for example, from 150 to 275 nanometers. Such particle sizes are measured with a laser granulometer.

An example of the aminosilicone corresponding to the formula set forth above is, for example, DC2-8299® from the company Dow Corning.

In one new embodiment, the at least one aminosilicone is used in the dyeing composition in an amount ranging from 0.01 to 20% by weight of the total weight of the composition. For example, this amount may range from 0.1 to 15% by weight and further, for example, from 0.5 to 10% by weight relative to the total weight of the composition.

Oxidation Dyes

The oxidation dyes may be chosen from oxidation bases and couplers.

In one embodiment, the compositions can comprise at least one oxidation base.

The oxidation bases which can be used are chosen from those conventionally known in oxidation dyeing. Representative examples of the at least one oxidation base include ortho- and para-phenylenediamines, double bases, ortho-aminophenols, para-aminophenols, heterocyclic bases and their acid addition salts.

For example, mention may be made of the following:

(A) para-phenylenediamines chosen from compounds of formula (II) and their acid addition salts:

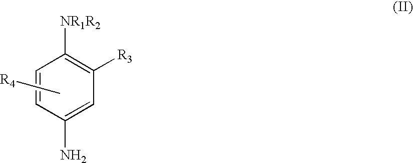

wherein:

R₁ is chosen from hydrogen, C₁-C₄ alkyl groups, C₁-C₄ monohydroxyalkyl groups, C₂-C₄ polyhydroxyalkyl groups, (C₁-C₄)alkoxy(C₁-C₄)alkyl groups, C₁-C₄ alkyl groups substituted with at least one group chosen from nitrogen-containing groups, phenyl groups and 4′-aminophenyl radicals;

R₂ is chosen from hydrogen, C₁-C₄ alkyl groups, C₁-C₄ monohydroxyalkyl groups, C₂-C₄ polyhydroxyalkyl radicals, (C₁-C₄)alkoxy(C₁-C₄)alkyl groups, and C₁-C₄ alkyl groups substituted with at least one group chosen from nitrogen-containing groups;

R₁ and R₂ may also form, with the nitrogen to which they are bonded, a 5- or 6-membered nitrogen-containing heterocycle ring, optionally substituted by at least one group chosen from alkyl groups, hydroxyl groups and ureido groups;

R₃ is chosen from hydrogen, halogens such as chlorine, C₁-C₄ alkyl groups, sulpho groups, carboxyl groups, C₁-C₄ monohydroxyalkyl groups, C₁-C₄ hydroxyalkoxy groups, C₁-C₄ acetylaminoalkoxy radicals, C₁-C₄ mesylaminoalkoxy radicals, and C₁-C₄ carbamoylaminoalkoxy radicals, and

R₄ is chosen from hydrogen, halogens, and C₁-C₄ alkyl radicals.

The nitrogen-containing groups of the formula (II) above may be chosen from, for example, amino, mono(C₁-C₄)alkylamino, di(C₁-C₄)alkylamino, tri(C₁-C₄)alkylamino, monohydroxy(C₁-C₄)alkylamino, imidazolinium, and ammonium radicals.

Representative para-phenylenediamines of formula (II) above may include, for example, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 2-methyl-1-N-β-hydroxyethyl-para-phenylenediamine, and their acid addition salts.

In one embodiment, the para-phenylenediamines of formula (II) above may, for example, be chosen from para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para- phenylenediamine, 2,3-dimethyl-para-phenylenediamine, N,N-bis(β-hydroxyethyl)-para-phenylenediamine, 2-chloro-para-phenylenediamine and their acid addition salts.

(B) According to one new embodiment, double bases are understood to mean compounds comprising at least two aromatic nuclei which carry at least one functional group chosen from amino groups and hydroxyl groups.

Representative double bases which can be used as the at least one oxidation base in the dyeing compositions may include doubles bases chosen from compounds of formula (III) and their acid addition salts:

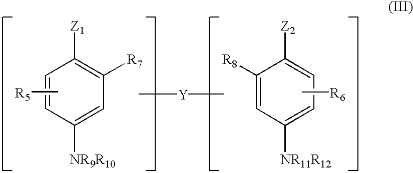

wherein:

Z₁ and Z₂, which may be identical or different, are each chosen from hydroxyl groups and —NH₂ radicals which may be substituted by C₁-C₄ alkyl radicals and by linking arm Y;

the linking arm Y is chosen from linear and branched, alkylene groups comprising from 1 to 14 carbon atoms, optionally interrupted by, or optionally terminating with, at least one entity chosen from nitrogen-containing groups and heteroatoms such as oxygen, sulphur and nitrogen, and optionally substituted by at least one group chosen from hydroxyl groups and C₁-C₆ alkoxy radicals;

R₅ and R₆, which may be identical or different, are each chosen from hydrogen, halogens, C₁-C₄ alkyl groups, C₁-C₄ monohydroxyalkyl groups, C₂-C₄ polyhydroxyalkyl groups, C₁-C₄ aminoalkyl radicals and linking arm Y;

R₇, R₈, R₉, R₁₀, R₁₁ and R₁₂, which may be identical or different, are each chosen from hydrogen, linking arm Y and C₁-C₄ alkyl radicals;

subject to the proviso that the compounds of formula (III) comprise only one linking arm Y per molecule.

Suitable nitrogen-containing groups of formula (III) may include, for example, amino, mono(C₁-C₄)alkylamino, di(C₁-C₄)alkylamino, tri(C₁-C₄)alkylamino, monohydroxy(C₁-C₄)alkylamino, imidazolinium, and ammonium radicals.

Representative double bases of formula (III) may include, for example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenol)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and their acid addition salts.

In one embodiment, the double bases of formula (III) may, for example, be chosen from N,N′-bis(β-hydroxyethyl)-N,N′-bis(4′-aminophenyl)-1,3-diaminopropanol, 1,8-bis(2,5-diaminophenoxy)-3,5-dioxaoctane, and their acid addition salts.

(C) the para-aminophenols can be chosen from compounds of formula (IV), and their acid addition salts:

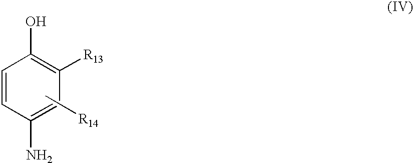

wherein:

R₁₃ is chosen from hydrogen, halogens, such as fluorine, C₁-C₄ alkyl groups, C₁-C₄ monohydroxyalkyl groups, (C₁-C₄)alkoxy(C₁-C₄)alkyl groups, C₁-C₄ aminoalkyl groups, and C₁-C₄ hydroxy(C₁-C₄)alkylaminoalkyl radicals and

R₁₄ is chosen from hydrogen, halogens, such as fluorine, C₁-C₄ alkyl groups, C₁-C₄ monohydroxyalkyl groups, C₂-C₄ polyhydroxyalkyl groups, C₁-C₄ aminoalkyl groups, C₁-C₄ cyanoalkyl groups, and (C₁-C₄)alkoxy(C₁-C₄)alkyl radicals.

Representative para-aminophenols of formula (IV) may include, for example, para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethyl-aminomethyl)phenol, and their acid addition salts.

(D) ortho-aminophenols which can be used as the at least one oxidation base may be chosen, for example, from 2-aminophenol, 2-amino-1-hydroxy-5-methylbenzene, 2-amino-1-hydroxy-6-methylbenzene, 5-acetamido-2-aminophenol, and their acid addition salts.

(E) heterocyclic bases which can be used as the at least one oxidation base in the dyeing compositions may be chosen, for example, from pyridine derivatives, pyrimidine derivatives, pyrazole derivatives, and their acid addition salts.

Representative pyridine derivatives may include the compounds described, for example, in Patent Nos. GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their acid addition salts.

Representative pyrimidine derivatives may include the compounds described in German Patent No. DE 2 359 399 or Japanese Patent Nos. JP 88-169 571 and JP 91-10659 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine and the pyrazolopyrimidine derivatives such as those mentioned in the patent application FR-A-2 750 048 and including pyrazolo[1,5-a]pyrimidine-3,7-diamine; 2,5-pyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)-(2-hydroxyethyl)amino]ethanol; 2-[(7-aminopyrazolo[1,5-a]pyrimidin-3-yl)-(2-hydroxyethyl)amino]ethanol; 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine; 3-amino-5-methyl-7-imidazolylpropylamino-pyrazolo[1,5-a]pyrimidine; and their addition salts, such as their acid addition salts, and their tautomeric forms where a tautomeric equilibrium exists.

Representative pyrazole derivatives may include the compounds described, for example, in Patent Nos. DE 3 843 892 and DE 4 133 957 and patent applications WO 94/08969, WO 94/08970, FR-A-2 733 749 and DE 195 43 988. Such pyrazole derivatives may include 4,5-diamino-1-methylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino- 1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methyl pyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their acid addition salts.

The at least one oxidation base can be present in an amount ranging, for example, from 0.0005 to 12% by weight, relative to the total weight of the composition, and further, for example, from 0.005 to 8% by weight, relative to the total weight of the composition.

In another embodiment, the compositions can comprise at least one coupler. The at least one coupler which can be used may be chosen from those which are conventionally used in oxidation dyeing compositions. The at least one coupler can be chosen, for example, from meta-aminophenols, meta-phenylenediamines, meta-diphenols, naphthols and heterocyclic couplers such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and their acid addition salts.

In one embodiment, the at least one coupler may, for example, be chosen from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindoline, 2,6-dihydroxy-4-methylpyridine, 1H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole and their acid addition salts.

The at least one coupler can be present in the dyeing composition in an amount ranging from 0.0001 to 10% by weight, relative to the total weight of the composition, and further, for example, from 0.005 to 5% by weight, relative to the total weight of the composition.

The acid addition salts of the at least one oxidation base and the at least one coupler may be chosen from hydrochlorides, hydrobromides, sulphates, tartrates, lactates and acetates.

Direct Dyes

The at least one direct dye which can be used in accordance with this disclosure may be chosen, for example, from neutral, acidic and cationic nitro benzene direct dyes, neutral, acidic and cationic azo direct dyes, neutral, acidic and cationic quinines, and further, for example, from anthraquinone direct dyes, azine direct dyes, triarylmethane direct dyes, indoamine direct dyes and natural direct dyes.

Non-limiting representatives of benzene direct dyes which can be used in accordance with this disclosure include the following compounds:

1,4-diamino-2-nitrobenzene

1-amino-2-nitro-4-β-hydroxyethylaminobenzene

1-amino-2-nitro-4-bis(β-hydroxyethyl)aminobenzene

1,4-bis(β-hydroxyethylamino)-2-nitrobenzene

1-β-hydroxyethylamino-2-nitro-4-bis(β-hydroxyethylamino)benzene

1-β-hydroxyethylamino-2-nitro-4-aminobenzene

1-β-hydroxyethylamino-2-nitro-4-(ethyl)(β-hydroxyethyl)aminobenzene

1-amino-3-methyl-4-β-hydroxyethylamino-6-nitrobenzene

1-amino-2-nitro-4-β-hydroxyethylamino-5-chlorobenzene

1,2-diamino-4-nitrobenzene

1-amino-2-β-hydroxyethylamino-5-nitrobenzene

1,2-bis(β-hydroxyethylamino)-4-nitrobenzene

1-amino-2-tris(hydroxymethyl)methylamino-5-nitrobenzene

1-hydroxy-2-amino-5-nitrobenzene

1-hydroxy-2-amino-4-nitrobenzene

1-hydroxy-3-nitro-4-aminobenzene

1-hydroxy-2-amino-4,6-dinitrobenzene

1-β-hydroxyethyloxy-2-β-hydroxyethylamino-5-nitrobenzene

1-methoxy-2-β-hydroxyethylamino-5-nitrobenzene

1-β-hydroxyethyloxy-3-methylamino-4-nitrobenzene

1-β,γ-dihydroxypropyloxy-3-methylamino-4-nitrobenzene

1-β-hydroxyethylamino-4-β,γ-dihydroxypropyloxy-2-nitrobenzene

1-β,γ-dihydroxypropylamino-4-trifluoromethyl-2-nitrobenzene

1-β-hydroxyethylamino-4-trifluoromethyl-2-nitrobenzene

1-β-hydroxyethylamino-3-methyl-2-nitrobenzene

1-β-aminoethylamino-5-methoxy-2-nitrobenzene

1-hydroxy-2-chloro-6-ethylamino-4-nitrobenzene

1-hydroxy-2-chloro-6-amino-4-nitrobenzene

1-hydroxy-6-bis(β-hydroxyethyl)amino-3-nitrobenzene

1-β-hydroxyethylamino-2-nitrobenzene

1-hydroxy-4-β-hydroxyethylamino-3-nitrobenzene.

The azo direct dyes which can be used in accordance with this disclosure include the cationic azo dyes described in the patent applications WO 95/15144, WO 95/01772 and EP-714954.

Representative azo direct dyes include the following:

1,3-dimethyl-2-[[4-(dimethylamino)phenyl]azo]-1H-imidazolium chloride,

1,3-dimethyl-2-[(4-aminophenyl)azo]-1H-imidazolium chloride and

1-methyl-4-[(methylphenylhydrazono)methyl]pyridinium methyl sulphate.

The azo direct dyes may further include, for example, the following dyes described in the Color Index International, 3rd edition:

Disperse Red 17

Acid Yellow 9

Acid Black 1

Basic Red 22

Basic Red 76

Basic Yellow 57

Basic Brown 16

Acid Yellow 36

Acid Orange 7

Acid Red 33

Acid Red 35

Basic Brown 17

Acid Yellow 23

Acid Orange 24

Disperse Black 9.

Further, the azo direct dyes may also include 1-(4′-aminodiphenylazo)-2-methyl-4-bis(β-hydroxyethyl)aminobenzene and 4-hydroxy-3-(2-methoxyphenylazo)-1-naphthalenesulphonic acid.

The quinone direct dyes may include the following

Disperse Red 15

Solvent Violet 13

Acid Violet 43

Disperse Violet 1

Disperse Violet 4

Disperse Blue 1

Disperse Violet 8

Disperse Blue 3

Disperse Red 11

Acid Blue 62

Disperse Blue 7

Basic Blue 22

Disperse Violet 15

Basic Blue 99

and may also include the following compounds:

1-N-methyl morpholiniumpropylamino-4-hydroxyanthraquinone

1-aminopropylamino-4-methylaminoanthraquinone

1-aminopropylaminoanthraquinone

5-β-hydroxyethyl-1,4-diaminoanthraquinone

2-aminoethylaminoanthraquinone

1,4-bis(β,γ-dihydroxypropylamino)anthraquinone.

The azine dyes may include the following compounds:

Basic Blue 17

Basic Red 2.

The triarylmethane dyes which may be used in accordance with this disclosure include the following compounds:

Basic Green 1

Acid Blue 9

Basic Violet 3

Basic Violet 14

Basic Blue 7

Acid Violet 49

Basic Blue 26

Acid Blue 7.

The indoamine dyes which can be used in accordance with this disclosure include the following compounds:

2-β-hydroxyethylamino-5-[bis-(β-4′-hydroxyethyl)amino]anilino-1,4-benzoquinone

2-β-hydroxyethylamino-5-(2′-methoxy-4′-amino)anilino-1,4-benzoquinone

3-N-(2′-chloro-4′-hydroxy)phenylacetylamino-6-methoxy-1,4-benzoquinoneimine

3-N-(3′-chloro-4′-methylamino)phenylureido-6-methyl-1,4-benzoquinoneimine

3-[4′-N-(ethylcarbamylmethyl)amino]phenylureido-6-methyl-1,4-benzoquinoneimine.

The natural direct dyes which can be used in accordance with this disclosure include lawsone, juglone, alizarin, purpurin, carminic acid, kermesic acid, purpurogallin, protocatechaldehyde, indigo, isatin, curcumin, spinulosin and apigenidin. It may also be possible to use extracts or decoctions comprising these natural dyes and, for example, cataplasms or extracts based on henna.

The at least one direct dye is present in the ready-to-use composition in an amount ranging from 0.001 to 20% by weight, relative to the total weight of the composition and further, for example, from 0.005 to 10% by weight, relative to the total weight of the composition.

The compositions may, for example, comprise at least one surfactant. The at least one surfactant may be chosen from anionic, amphoteric, nonionic, zwifterionic and cationic surfactants.

The at least one surfactant employed can be, for example, chosen from:

(i) Anionic Surfactants:

As non-limiting examples of anionic surfactants which can be used, alone or as mixtures, mention may be made, for example, of salts such as alkali metal salts, for example, sodium salts, ammonium salts, amine salts, amino alcohol salts and magnesium salts of the following compounds: alkyl sulphates, alkyl ether sulphates, alkylamido ether sulphates, alkylaryl polyether sulphates, monoglyceride sulphates; alkylsulphonates, alkyl phosphates, alkylamidesulphonates, alkylarylsulphonates, α-olefinsulphonates, paraffinsulphonates; (C₆-C₂₄)alkyl sulphosuccinates, (C₆-C₂₄)alkyl ether sulphosuccinates, (C₆-C₂₄)alkylamide sulphosuccinates; (C₆-C₂₄)alkyl sulphoacetates; (C₆-C₂₄)acyl sarcosinates and (C₆-C₂₄)acyl glutamates. It is also possible to use the carboxylic esters of (C₆-C₂₄)alkyl polyglycosides, such as alkylglucoside citrates, alkylpolyglycoside tartrates and alkylpolyglycoside sulphosuccinates, alkylsulphosuccinamates; acyl isethionates and N-acyltaurates. The alkyl or acyl radical of all these different compounds comprises, for example, from 12 to 20 carbon atoms, and the aryl radical is chosen, for example, from phenyl and benzyl groups. Among the anionic surfactants which can be used, mention may also be made of fatty acid salts such as the salts of oleic, ricinoleic, palmitic and stearic acids, coconut oil acid and hydrogenated coconut oil acid; and acyl lactylates wherein the acyl radical comprises from 8 to 20 carbon atoms. It is also possible to use alkyl-D-galactosideuronic acids and their salts, polyoxyalkylenated (C₆-C₂₄)alkyl ether carboxylic acids, polyoxyalkylenated (C₆-C₂₄)alkyl aryl ether carboxylic acids, polyoxyalkylenated (C₆-C₂₄)alkylamido ether carboxylic acids and their salts, for example those comprising from 2 to 50 alkylene oxide groups, for example ethylene oxide groups, and mixtures thereof.

(ii) Nonionic Surfactants:

The nonionic surfactants are compounds that are well known (see, for example, in this respect “Handbook of Surfactants” by M. R. Porter, published by Blackie & Son (Glasgow and London), 1991, pp. 116-178) and, in the context of the present disclosure, their nature is not a critical feature. They can be chosen, for example, from polyethoxylated and polypropoxylated alkylphenols, alpha-diols and alcohols comprising a fatty chain comprising, for example, 8 to 18 carbon atoms, it being possible for the number of ethylene oxide or propylene oxide groups to range, for example, from 2 to 50. Mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising on average from 1 to 5, such as 1.5 to 4, glycerol groups; polyethoxylated fatty amines such as those comprising from 2 to 30 mol of ethylene oxide; oxyethylenated fatty acid esters of sorbitan comprising from 2 to 30 mol of ethylene oxide; fatty acid esters of sucrose, fatty acid esters of polyethylene glycol, alkyl polyglycosides, N-alkylglucamine derivatives, amine oxides such as (C₁₀-C₁₄)alkylamine oxides or N-acylaminopropylmorpholine oxides.

(iii) Amphoteric or Zwitterionic Surfactants:

The amphoteric or zwitterionic surfactants, whose nature is not a critical feature in the context of the present disclosure, can be chosen, for example, from aliphatic secondary and tertiary amine derivatives wherein the aliphatic radical is chosen from linear and branched chains comprising 8 to 18 carbon atoms and comprising at least one water-solubilizing anionic group (for example carboxylate, sulphonate, sulphate, phosphate or phosphonate); mention may also be made of (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines.

Among the amine derivatives, mention may be made of the products sold under the name Miranol, as described in U.S. Pat. Nos. 2,528,378 and 2,781,354 and classified in the CTFA dictionary, 3rd edition, 1982, under the names amphocarboxyglycinates and amphocarboxypropionates of respective structures of:

R₂—CONHCH₂CH₂—N⁺(R₃)(R₄)(CH₂COO⁻)

wherein: R₂ is chosen from alkyl radicals derived from an acid R₂—COOH present in hydrolysed coconut oil, and heptyl, nonyl and undecyl radicals, R₃ is a beta-hydroxyethyl group and R₄ is a carboxymethyl group; and of

R₂′—CONHCH₂CH₂—N(B)(C)

wherein:

B represents —CH₂CH₂OX′, C represents —(CH₂)_{z}—Y′, with z=1 or 2,

X′ is chosen from —CH₂CH₂—COOH and a hydrogen atom,

Y′ is chosen from —COOH and —CH₂—CHOH—SO₃H radicals,

R₂′ is chosen from alkyl radicals of an acid R₉—COOH present in coconut oil or in hydrolysed linseed oil, alkyl radicals, such as C₇, C₉, C₁₁ and C₁₃ alkyl radicals, a C₁₇ alkyl radical and its iso form, and an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold under the trade name Miranol® C2M concentrate by the company Rhodia Chimie.

(iv) Cationic Surfactants:

The cationic surfactants, may be chosen, for example, from: salts of optionally polyoxyalkylenated primary, secondary and tertiary fatty amine; quaternary ammonium salts such as tetraalkylammonium, alkylamidoalkyltrialkylammonium, trialkylbenzylammonium, trialkylhydroxyalkylammonium and alkylpyridinium chlorides and bromides; imidazoline derivatives; and amine oxides of cationic nature.

The amount of surfactants present in the composition can range from 0.01 to 40% by weight, such as from 0.5 to 30% by weight, relative to the total weight of the composition.

Medium

The medium for the composition can be, for example, an aqueous medium comprising water and can, for example, further comprise cosmetically acceptable organic solvents such as, for example, alcohols such as ethyl alcohol, isopropyl alcohol, benzyl alcohol and phenylethyl alcohol or glycols or glycol ethers such as ethylene glycol monomethyl, monoethyl and monobutyl ethers, propylene glycol or its ethers such as propylene glycol monomethyl ether, butylene glycol, dipropylene glycol and also the alkyl ethers of diethylene glycol, such as diethylene glycol monoethyl and monobutyl ether. The organic solvents may therefore be present in a concentration ranging from 0.5 to 20% and, for example, from 2 to 10% by weight relative to the total weight of the composition.

Ingredient Agents

The compositions may further comprise rheology modifiers such as cellulosic thickeners (hydroxyethylcellulose, hydroxypropylcellu lose, carboxymethylcellulose, etc.), guar gum and its derivatives (hydroxypropylguar, etc.), gums of microbial origin (xanthan gum, scleroglucan gum, etc.), and synthetic thickeners such as crosslinked homopolymers of acrylic acid or of acrylamidopropanesulphonic acid.

The composition may further comprise at least one ionic or nonionic associative polymer selected, for example, from the polymers sold under the names Pemulen® TR1 and TR2 by the company Goodrich, Salcare SC90® by the company Allied Colloids, Aculyn® 22, 28, 33, 44 or 46 by the company Rohm & Haas and Elfacos® T210 and T212 by Akzo, in an amount ranging, for example, from 0.01 to 10% by weight relative to the total weight of the composition.

Further, the compositions can also comprise at least one cationic or amphoteric conditioning polymer which is well known in the art in the field of dyeing of human keratin fibers, in an amount ranging from 0.01 to 10% by weight relative to the total weight of the composition, for example, from 0.05 to 5% and further, for example, from 0.1 to 3% by weight relative to the total weight of the composition.

Cationic Polymers

As used herein, the expression “cationic polymer” means any polymer comprising cationic groups and/or groups that can be ionized into cationic groups.

The cationic polymers that can be used may be chosen from any of those already known by those skilled in the art as improving at least one cosmetic property of the hair, for example, those described in patent application EP-A-337 354 and in French Patent Nos. FR-2 270 846, 2 383 660, 2 598 611, 2 470 596 and 2 519 863.

The cationic polymers may, for example, be chosen from those comprising units comprising at least one amine group chosen from primary, secondary, tertiary and quaternary amine groups which can either form part of the main polymer chain or are borne by a side substituent directly attached to the main polymer chain.

The cationic polymers generally have a number-average molecular mass ranging, for example, from 500 to 5×10⁶ and further, for example, from 10³ to 3×10⁶.

Among the cationic polymers, mention may be made, for example, of polymers of polyquaternary ammonium, polymers of polyamino amide and polymers of polyamine. These polymers are known in the art.

The polymers of polyamine, polymers of polyamino amide and polymers of polyquarternary ammonium are described, for example, in French Patents Nos. 2 505 348 or 2 542 997. Among these polymers, mention may be made of:

(1) homopolymers or copolymers derived from acrylic or methacrylic esters or amides and comprising at least one of the units chosen from the units of formula (I), (II), (III) and (IV) below:

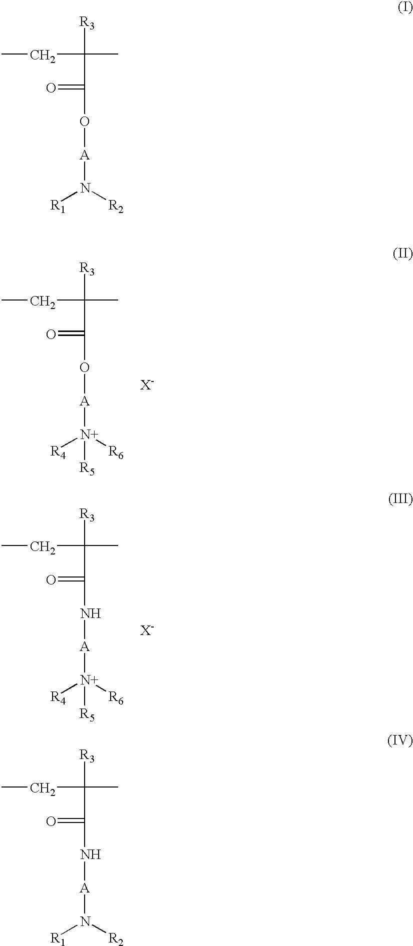

wherein:

R₃, which may be identical or different, is chosen from a hydrogen atom and a CH₃ radical;

A, which may be identical or different, is chosen from linear and branched alkyl groups of 1 to 6 carbon atoms, such as 2 or 3 carbon atoms, and a hydroxyalkyl groups of 1 to 4 carbon atoms;

R₄, R₅ and R₆, which may be identical or different, are chosen from alkyl groups comprising from 1 to 18 carbon atoms, for example, alkyl groups comprising from 1 to 6 carbon atoms and benzyl radicals.

R₁ and R₂, which are identical or different, are chosen from hydrogen and alkyl groups comprising from 1 to 6 carbon atoms, for example, methyl and ethyl groups;

X⁻ is an anion derived from an inorganic or organic acid, such as a methosulphate anion or an anion chosen from halides such as chloride and bromide.

The polymers of family (1) can also comprise at least one unit derived from comonomers that may be chosen from the family of acrylamides, methacrylamides, diacetoneacrylamides, acrylamides and methacrylamides substituted on the nitrogen with at least one group chosen from lower (C₁-C₄) alkyls, acrylic, methacrylic acids, acrylic esters, methacrylic esters, and vinyllactams such as vinylpyrrolidone, vinylcaprolactam, and vinyl esters.

Thus, among these polymers of class (1), mention may be made of:

the copolymers of acrylamide and of dimethylaminoethyl methacrylate quaternized with dimethyl sulphate or with a dimethyl halide, such as the product sold under the name Hercofloc by the company Hercules,

the copolymers of acrylamide and of methacryloyloxyethyltrimethylammonium chloride described, for example, in patent application EP-A-080 976 and sold under the name Bina Quat P 100 by the company Ciba Geigy,

the copolymer of acrylamide and of methacryloyloxyethyltrimethylammonium methosulphate sold under the name Reten by the company Hercules,

quaternized and non-quaternized vinylpyrrolidone/dialkylaminoalkyl acrylate or methacrylate copolymers, such as the products sold under the name Gafquat by the company ISP, for example, Gafquat 734 or Gafquat 755, or the products known as “Copolymer 845, 958 and 937.” These polymers are described in detail in French Patent Nos. 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylamidopropyidimethylamine copolymers sold, for example, under the name Styleze CC 10 by the company ISP, and

quaternized vinylpyrrolidone/dimethylaminopropylmethacrylamide copolymers such as the product sold under the name Gafquat HS 100 by the company ISP.

(2) The cellulose ether derivatives comprising quaternary ammonium groups, described in French Patent No.1 492 597 and, for example, the polymers sold under the names JR (JR 400, JR 125 and JR 30M) or LR (LR 400, or LR 30M) by the company Union Carbide Corporation. These polymers are also defined in the CTFA dictionary as quaternary ammoniums of hydroxyethylcellulose that has reacted with an epoxide substituted by a trimethylammonium group.

(3) The cationic cellulose derivatives such as cellulose copolymers or cellulose derivatives grafted with a water-soluble monomer of quaternary ammonium, described, for example, in U.S. Pat. No. 4,131,576, such as hydroxyalkylcelluloses, for instance, hydroxymethylcelluloses, hydroxyethylcelluloses or hydroxypropylcelluloses grafted, for example, with a salt chosen from methacryloylethyltrimethylammonium salts, methacrylamidopropyltrimethylammonium salts and dimethyldiallylammonium salts.

The commercial products corresponding to this definition are, for example, the products sold under the names Celquat L 200 and Celquat H 100 by the company National Starch.

(4) The cationic polysaccharides described, for example, in U.S. Pat. Nos. 3,589,578 and 4,031,307, such as guar gums comprising cationic trialkylammonium groups. For example, guar gums modified with a salt (e.g. chloride) of 2,3-epoxypropyltrimethylammonium may be used.

Such products are sold, for example, under the trade names Jaguar C13 S, Jaguar C 15, Jaguar C 17 or Jaguar C162 by the company Meyhall.

(5) Polymers comprising piperazinyl units and divalent alkylene or hydroxyalkylene radicals comprising straight or branched chains, optionally interrupted by at least one atom chosen from oxygen, sulphur and nitrogen atoms or by at least one aromatic or heterocyclic ring, and also at least one of the oxidation and/or quaternization products of these polymers. Such polymers are described, for example, in French Patent Nos. 2 162 025 and 2 280 361.

(6) Water-soluble polyamino amides prepared, for example, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative. The crosslinking agent can be used in proportions ranging from 0.025 to 0.35 mol per amine group of the polyamino amide. These polyamino amides can be alkylated or, if they comprise at least one tertiary amine function, they can be quaternized. Such polymers are described, for example, in French patents 2 252 840 and 2 368 508.

(7) The polyamino amide derivatives resulting from the condensation of polyalkylene polyamines with polycarboxylic acids followed by alkylation with difunctional agents. Mention may be made, for example, of adipic acid/diacylaminohydroxyalkyl-dialkylenetriamine polymers wherein the alkyl radical comprises from 1 to 4 carbon atoms and, for example, may be chosen from methyl, ethyl and propyl groups. Such polymers are described, for example, in French Patent No.1 583 363.

Among these derivatives, mention may be made, for example, of the adipic acid/dimethylaminohydroxypropyl/diethylenetriamine polymers sold under the name Cartaretine F, F4 or F8 by the company Sandoz.

(8) The polymers obtained by reaction of a polyalkylene polyamine comprising two primary amine groups and at least one secondary amine group with a dicarboxylic acid chosen from diglycolic acids and saturated aliphatic dicarboxylic acids comprising from 3 to 8 carbon atoms. The molar ratio between the polyalkylene polyamine and the dicarboxylic acid may range, for example, from 0.8:1 to 1.4:1; the polyamino amide resulting therefrom may be reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.

Other non-limiting examples of such derivatives include the adipic acid/epoxypropyl/diethylenetriamine copolymers sold, for example, under the name Hercosett 57 by the company Hercules Inc. or under the name PD 170 or Delsette 101 by the company Hercules.

(9) Cyclopolymers of alkyldiallylamine or of dialkyldiallylammonium, such as the homopolymers or copolymers comprising, as a main constituent of the chain, at least one of the units corresponding to the formula (V) or (VI):

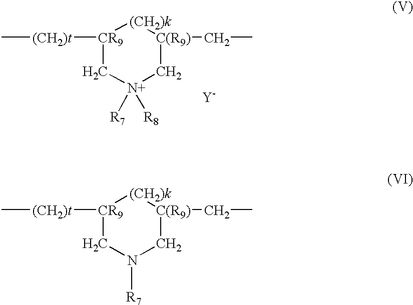

wherein k and t are equal to 0 or 1, the sum k+t being equal to 1;

R₉ is chosen from a hydrogen atom and a methyl radical;

R₇ and R₈, which may be identical or different, are chosen from alkyl groups, comprising from 1 to 8 carbon atoms, hydroxyalkyl groups in which the alkyl group, for example, comprises from 1 to 5 carbon atoms, and lower (C₁-C₄)amidoalkyl groups, or R₇ and R₈ can be chosen from, together with the nitrogen atom to which they are attached, heterocyclic groups such as piperidyl or morpholinyl; in one embodiment, R₇ and R₈, which may be identical or different, can be chosen from alkyl groups comprising from 1 to 4 carbon atoms; Y⁻ is an anion such as bromide, chloride, acetate, borate, citrate, tartrate, bisulphate, bisulphite, sulphate and phosphate. These polymers are described, for example, in French Patent No. 2 080 759 and in its Certificate of Addition 2 190 406.

Among the polymers defined above, mention may be made, for example, of the dimethyldiallylammonium chloride homopolymer sold under the name “Merquat 100” by the company Calgon (andits homologues of low weight-average molecular mass) and copolymers of diallyldimethylammonium chloride and of acrylamide, sold under the name “Merquat 550.”

(10) The quaternary diammonium polymer comprising repeating units corresponding to the formula (VII):

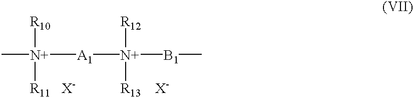

wherein:

R₁₀, R₁₁, R₁₂ and R₁₃, which may be identical or different, are chosen from aliphatic radicals, alicyclic radicals and arylaliphatic radicals comprising from 1 to 20 carbon atoms and from lower hydroxyalkylaliphatic radicals, or R₁₀, R₁₁, R₁₂ and R₁₃, together or separately, constitute, with the nitrogen atoms to which they are attached, heterocycles optionally comprising a second heteroatom other than nitrogen, or R₁₀, R₁₁, R₁₂ and R₁₃ are chosen from linear and branched C₁-C₆ alkyl radicals substituted with at least one group chosen from nitrile, ester, acyl and amide groups and groups of formulae —CO—O—R₁₄-D and —CO—NH—R₁₄-D wherein R₁₄ is chosen from alkylene groups and D is chosen from quaternary ammonium groups;

A₁ and B₁, which may be identical or different, are chosen from linear and branched, saturated and unsaturated polymethylene groups comprising from 2 to 20 carbon atoms. The polymethylene groups may comprise, linked to or intercalated in the main chain, at least one entity chosen from aromatic rings, oxygen, sulphur atoms, and sulphoxide, sulphone, disulphide, amino, alkylamino, hydroxyl, quaternary ammonium, ureido, amide and ester groups, and

X⁻ is an anion chosen from anions derived from inorganic and organic acids;

A₁, R₁₀ and R₁₂ may optionally form, with the two nitrogen atoms to which they are attached, a piperazine ring. In addition, if A₁ is a radical chosen from linear and branched, saturated and unsaturated alkylene and hydroxyalkylene radicals, B₁ can also denote a group —(CH₂)ₙ—CO-D-OC—(CH₂)ₙ— wherein n ranges from 1 to 100, such as from 1 and 50.

D is chosen from:

a) a glycol residue of formula: —O-Z-O—, wherein Z is chosen from linear and branched hydrocarbon-based radicals and a group corresponding to one of the following formulae:

—(CH₂—CH₂—O)ₓ—CH₂—CH₂— and

—[CH₂—CH(CH₃)—O]_{y}—CH₂—CH(CH₃)—

wherein x and y, which may be identical or different, are each an integer ranging from 1 to 4, representing a defined and unique degree of polymerization or any number ranging from 1 to 4 representing an average degree of polymerization;

b) a bis-secondary diamine residue such as a piperazine derivative;

c) a bis-primary diamine residue of formula: —NH—Y—NH—, wherein Y is chosen from linear and branched hydrocarbon radicals, and the divalent radical

—CH₂—CH₂—S—S—CH₂—CH₂—; and

d) a ureylene group of formula: —NH—CO—NH—.

In one embodiment, X⁻ is an anion such as chloride or bromide.

These polymers may have a number-average molecular mass ranging from 1000 to 100,000.

These polymers are described, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Further, the polymers that comprise repeating units corresponding to the formula (VIII) below can be used:

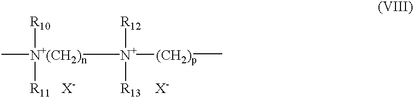

wherein R₁₀, R₁₁, R₁₂ and R₁₃, which are identical or different, are chosen from alkyl and hydroxyalkyl radicals comprising from 1 to 4 carbon atoms, n and p, which may be identical or different, are integers ranging from 2 to 20, and X⁻ is an anion chosen from anions derived from mineral and organic acids.

(11) Poly(quaternary ammonium) polymers comprising repeating units of formula (IX):

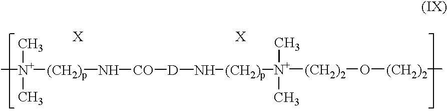

wherein p is an integer ranging from 1 to 6, D can be nothing or can represent a group —(CH₂)ᵣ—CO— in which r may be an integer ranging from 4 to 7, and X⁻ is an anion.

Such polymers may be prepared by the processes disclosed in U.S. Pat. Nos. 4,157,388, 4,702,906 and 4,719,282. They are described, for example, in patent application EP-A-122 324.

Among these polymers, mention may be made of the products “Mirapol A 15,” “Mirapol AD1,” “Mirapol AZ1” and “Mirapol 175,” which are sold by the company Miranol.

(12) Quaternary polymers of vinylpyrrolidone and of vinylimidazole, such as the products sold under the names Luviquat FC 905, FC 550 and FC 370 by the company BASF.

(13) Polyamines such as Polyquart H sold by Henkel, referenced under the name “Polyethylene glycol (15) tallow polyamine” in the CTFA dictionary.

(14) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, such as methylenebisacrylamide. A crosslinked acrylamide/methacryloyloxyethyltrimethylammonium chloride copolymer (20/80 by weight) in the form of a dispersion comprising 50% by weight of said copolymer in mineral oil can, for example, be used. This dispersion is sold under the name “Salcare® SC 92” by the company Allied Colloids. In another embodiment, a crosslinked methacryloyloxyethyltrimethylammonium chloride homopolymer comprising about 50% by weight of the homopolymer in mineral oil or in a liquid ester can also be used. These dispersions are sold under the names Salcare® SC 95 and Salcare® SC 96 by the company Allied Colloids.

Other cationic polymers can be chosen from polyalkyleneimines, for example, polyethyleneimines, polymers containing vinylpyridine and vinylpyridinium units, condensates of polyamines and of epichlorohydrin, quaternary polyureylenes and chitin derivatives.

Among all the cationic polymers that can be used, non-limiting examples include the polymers of families (1), (9), (10), (11) and (14) and, in one new embodiment, polymers comprising repeating units chosen from units of formulae (W) and (U) below can be used:

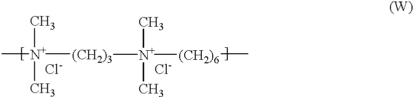

and, for example, those polymers comprising repeating units of formula (W) whose weight-average molar mass, determined by gel permeation chromatography, ranges from 9500 to 9900;

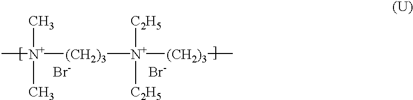

and, for example, those polymers comprising repeating units of formula (U) whose weight-average molar mass, determined by gel permeation chromatography, is about 1200.

Amphoteric Polymers

The amphoteric polymers may be chosen from polymers comprising units K and M distributed randomly in the polymer chain, in which K is a unit derived from a monomer comprising at least one basic nitrogen atom and M is a unit derived from an acidic monomer comprising at least one group chosen from carboxylic and sulphonic group, or K and M can be chosen from groups derived from zwitterionic carboxybetaine or sulphobetaine monomers.

K and M can also be chosen from a cationic polymer chain comprising at least one group chosen from primary, secondary, tertiary and quaternary amine groups, in which at least one of the amine groups bears a carboxylic or sulphonic group connected via a hydrocarbon-based radical, or alternatively K and M can form part of a chain of a polymer comprising an (α,β-dicarboxylic ethylene unit in which one of the carboxylic groups has been made to react with a polyamine comprising at least one amine chosen from primary and secondary amine groups.

The amphoteric polymers corresponding to the above definition, for example, are chosen from the following polymers:

(1) Polymers resulting from the copolymerization of at least one monomer derived from a vinyl compound bearing a carboxylic group such as, for example, acrylic acid, methacrylic acid, maleic acid, alpha-chloroacrylic acid, and at least one basic monomer derived from a substituted vinyl compound comprising at least one basic atom, such as dialkylaminoalkyl methacrylates and acrylates, dialkylaminoalkylmethacrylamides and -acrylamides. Such compounds are described, for example, in U.S. Pat. No. 3,836,537.

Mention may also be made of the sodium acrylate/acrylamidopropyl-trimethylammonium chloride copolymer sold under the name Polyquart KE 3033 by the company Henkel.

The vinyl compound can also be a dialkyldiallylammonium salt such as dimethyldiallylammonium chloride. The copolymers of acrylic acid and of the latter monomer are sold under the names Merquat 280, Merquat 295 and Merquat Plus 3330 by the company Calgon.

(2) Polymers comprising units deriving from:

a) at least one monomer selected from acrylamides and methacrylamides substituted on the nitrogen with an alkyl radical,

b) at least one acidic comonomer comprising at least one reactive carboxylic group, and

c) at least one basic comonomer such as esters comprising substituents chosen from primary, secondary, tertiary and quaternary amine substituents of acrylic and methacrylic acids and the product of quaternization of dimethylaminoethyl methacrylate with dimethyl or diethyl sulphate.

In one embodiment, the N-substituted acrylamides or methacrylamides are, for example, groups in which the alkyl radicals comprise from 2 to 12 carbon atoms, such as, N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen, for example, from acrylic acids, methacrylic acids, crotonic acids, itaconic acids, maleic acids and fumaric acids and alkyl monoesters, comprising 1 to 4 carbon atoms, of maleic or fumaric acids or anhydrides.

The basic comonomers are chosen, for example, from aminoethyl, butylaminoethyl, N,N′-dimethylaminoethyl and N-tert-butylaminoethyl methacrylates.

The copolymers having the CTFA (4th edition, 1991) name octylacrylamide/acrylates/butylaminoethyl methacrylate copolymer such as the products sold under the name Amphomer or Lovocryl 47 by the company National Starch can, for example, be used.

(3) crosslinked and alkylated polyamino amides partially or totally derived from polyamino amides of general formula:

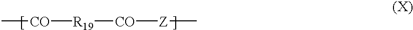

wherein R₁₉ is chosen from a divalent radical derived from a saturated dicarboxylic acid, mono- and dicarboxylic aliphatic acids comprising an ethylenic double bond, an ester of a lower alkanol, comprising 1 to 6 carbon atoms, of these acids and a radical derived from the addition of any one of the acids to amines chosen from bis(primary) or bis(secondary) amines, and Z is chosen from bis(primary), mono- and bis(secondary) polyalkylene-polyamine radicals and, for example, Z represents:

a) in proportions ranging from 60 to 100 mol %, the radical of formula (XI)

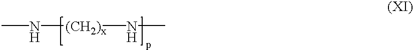

wherein x=2 and p=2 or 3, or x=3 and p=2 this radical being derived from a compound chosen from diethylenetriamine, triethylenetetraamine and dipropylenetriamine;

b) in proportions ranging from 0 to 40 mol %, the radical (XI) above in which x=2 and p=1 and which is derived from a compound chosen from ethylenediamine and piperazine:

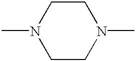

c) in proportions of from 0 to 20 mol %, the —NH—(CH₂)₆—NH— radical, which is derived from hexamethylenediamine, these polyamino amines can be crosslinked by addition of a difunctional crosslinking agent selected from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

In one embodiment, the saturated carboxylic acids are chosen from acids comprising 6 to 10 carbon atoms, such as adipic acid, 2,2,4-trimethyladipic acid and 2,4,4-trimethyladipic acid, terephthalic acid, acids comprising an ethylenic double bond such as, for example, acrylic acid, methacrylic acid and itaconic acid.

The alkane sultones used in the alkylation are chosen, for example, from propane sultone and butane sultone, the salts of the alkylating agents can be chosen, for example, from sodium and potassium salts.

(4) polymers comprising zwitterionic units of formula (XII):

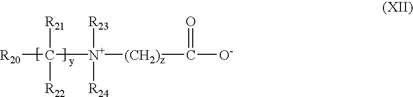

wherein R₂₀ is chosen from polymerizable unsaturated groups such as acrylate, methacrylate, acrylamide and methacrylamide groups, y and z, which may be identical or different, are chosen from integers ranging from 1 to 3, R₂₁, and R₂₂, which may be identical or different, are chosen from a hydrogen atom, methyl, ethyl and propyl groups, R₂₃ and R₂₄, which may be identical or different, are chosen from a hydrogen atom and alkyl radicals such that the sum of the carbon atoms in R₂₃ and R₂₄ does not exceed 10.

The polymers comprising such units can also comprise units derived from non-zwitterionic monomers such as monomers chosen from dimethyl and diethylaminoethyl acrylates, methacrylates, alkyl acrylates, acrylamides, methacrylamides, and vinyl acetates.

By way of example, mention may be made of the copolymer of butyl methacrylate/dimethyl-carboxymethylammonioethyl methacrylate such as the product sold under the name Diaformer Z301 by the company Sandoz.

(5) polymers derived from chitosan comprising monomer units corresponding to the following formulae (XIII), (XIV) and (XV) below:

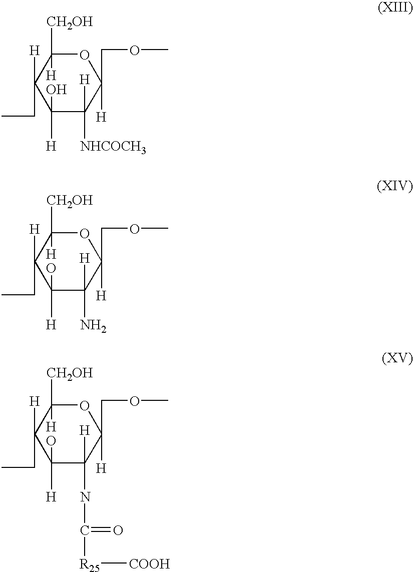

the unit (XIII) being present in proportions ranging from 0 to 30%, the unit (XIV) in proportions ranging from 5 and 50% and the unit (XV) in proportions ranging from 30 to 90%, and wherein in unit (XV), R₂₅ is a radical of formula:

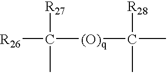

wherein q denotes 0 or 1;

if q=0, R₂₆, R₂₇ and R₂₈, which may be identical or different, are each chosen from a hydrogen atom, methyl, hydroxyl, acetoxy and amino residues, and monoalkylamine residues and dialkylamine residues, which are optionally interrupted by at least one nitrogen atom and/or optionally substituted with at least one group chosen from amine, hydroxyl, carboxyl, alkylthio and sulphonic groups, and alkylthio residues in which the alkyl group bears an amino residue, at least one of the radicals R₂₆, R₂₇ and R₂₈ being, in this case, a hydrogen atom;

or, if q=1, R₂₆, R₂₇ and R₂₈, which may be identical or different, are each chosen from a hydrogen atom, and salts formed by these compounds with bases or acids.

(6) polymers derived from the N-carboxyalkylation of chitosan, such as N-carboxymethylchitosan or N-carboxybutylchitosan sold under the name “Evalsan” by the company Jan Dekker.

(7) polymers corresponding to the general formula (XI) such as those described, for example, in French Patent No. 1 400 366:

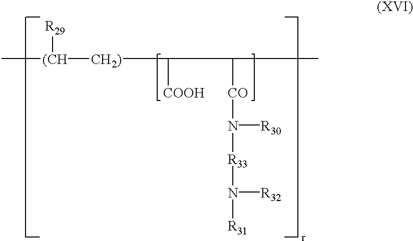

wherein R₂₉ is chosen from a hydrogen atom, and CH₃O, CH₃CH₂O and phenyl radicals, R₃₀ is chosen from a hydrogen and lower alkyl radicals such as methyl and ethyl, R₃₁ is chosen from a hydrogen and lower alkyl radicals such as methyl and ethyl, R₃₂ is chosen from lower alkyl radicals such as methyl and ethyl and radicals corresponding to the formula: —R₃₃—N(R₃₁)₂, wherein R₃₃ is chosen from —CH₂—CH₂—, —CH₂—CH₂—CH₂— and —CH₂—CH(CH₃)— groups, and R₃₁ is chosen from a hydrogen atom and lower alkyl radicals such as methyl and ethyl, and also the higher homologues of these radicals comprising up to 6 carbon atoms;

r is chosen such that the number-average molecular weight of said polymer ranges from 500 to 6,000,000, such as from 1000 to 1,000,000.

(8) amphoteric polymers of the type -D-X-D-X— chosen from:

a) polymers obtained by the action of chloroacetic acid or sodium chloroacetate on compounds comprising at least one unit of formula:

-D-X-D-X-D- (XVII)

wherein D is a radical

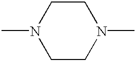

and X is chosen from the symbols E and E′, wherein E and E′, which may be identical or different, are chosen from divalent alkylene radicals comprising at least one chain chosen from straight and branched chains comprising up to 7 carbon atoms in the main chain, wherein said divalent alkylene radicals are optionally substituted with at least one hydroxyl group. E or E′ can additionally comprise at least one atom chosen from oxygen, nitrogen and sulphur atoms, and 1 to 3 rings chosen from aromatic and heterocyclic rings. The oxygen, nitrogen and sulphur atoms can be present in the form of at least one group chosen from ether, thioether, sulphoxide, sulphone, sulphonium, alkylamine and alkenylamine, hydroxyl, benzylamine, amine oxide, quaternary ammonium, amide, imide, alcohol, ester and urethane groups;

b) polymers of formula:

-D-X-D-X— (XVIII)

wherein D is a radical

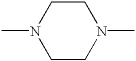

and X is chosen from the symbol E and E′ and wherein at least one X is chosen from E′; E having the meaning given above and E′ being a divalent radical chosen from divalent alkylene radicals comprising at least one chain chosen from straight and branched chains comprising up to 7 carbon atoms in the main chain, wherein said divalent alkylene radicals are optionally substituted with at least one hydroxyl radical and comprise at least one nitrogen atom substituted with an alkyl chain, which is optionally interrupted by an oxygen atom and further comprising at least one functional group chosen from carboxyl functional groups and hydroxyl functional groups which are betainized by reaction with a reactant chosen from chloroacetic acid and sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also contain other vinyl comonomers such as vinylcaprolactam.

In one new embodiment, the amphoteric polymers are those of family (1).

In another new embodiment, the composition may further comprise an effective amount of other agents known in direct or oxidation dyeing, such as various customary adjuvants, for example sequestrants such as EDTA and etidronic acid, UV filters, waxes, volatile or nonvolatile, cyclic or linear or branched, organically modified or unmodified silicones which are different from those disclosed herein, preservatives, ceramides, pseudoceramides, plant oils, mineral oils or synthetic oils, vitamins or provitamins such as panthenol, and opacifiers.

The composition may further comprise at least one agent chosen from reducing agents and antioxidants. Representative agents may be chosen from, for example, sodium sulphite, thioglycolic acid, thiolactic acid, sodium bisulphite, dehydroascorbic acid, hydroquinone, 2-methylhydroquinone, tert-butylhydroquinone and homogentisic acid. Such agents are generally present in an amount ranging from, for example, 0.05 to 3% by weight relative to the total weight of the composition.

The person skilled in the art will of course take care to select the optional complementary compound(s) mentioned above in such a way that the advantageous properties intrinsically attached to the dyeing composition are not, or not substantially, adversely affected by the intended addition or additions.

In the ready-to-use composition or in the oxidizing composition, the at least one oxidizing agent can, for example, be chosen from urea peroxide, alkali metal bromates, ferricyanides and persalts such as perborates and persulphates. In one embodiment, the at least one oxidizing agent can, for example, be hydrogen peroxide. When the at least one oxidizing agent is hydrogen peroxide, the at least one oxidizing agent may, for example, comprise a solution of oxygenated water with a titre ranging, for example, from 1 to 40 in volume, such as, for example from 5 to 40 in volume.

It is likewise possible to use, as the at least one oxidizing agent, at least one oxidation-reduction enzyme. The oxidation-reduction enzymes can be chosen from, for example, laccases, peroxidases and 2-electron oxidoreductases (such as uricase), where appropriate in the presence of their respective donor or cofactor.

The pH of the ready-to-use composition applied to the keratin fibers [composition resulting from the mixing of the at least one dyeing composition and the at least one oxidizing composition] ranges from 4 and 11, such as, for example, from 6 to 10. This pH may be adjusted to the desired value by means of at least one agent for adjusting pH chosen from acidifying and basifying agents which are well known in the state of the art in the dyeing of keratin fibers.

The basifying agents may be chosen from, by way of example, aqueous ammonia, alkali metal carbonates, alkanolamines such as mono-, di- and triethanolamines and also derivatives thereof, oxyethylenated ethylenediamines, oxypropylenated ethylenediamines, hydroxyalkylamines, ethylenediamines, sodium hydroxide, potassium hydroxide and the compounds of formula (V):

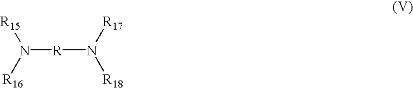

wherein R is a propylene residue optionally substituted with a group chosen from a hydroxyl group and C₁-C₄ alkyl groups;

R₁₅, R₁₆, R₁₇ and R₁₈, which are identical or different, may each be chosen from hydrogen, C₁-C₄ alkyl groups, and C₁-C₄ hydroxyalkyl groups.

The acidifying agents are conventionally, by way of example, chosen from mineral acids and organic acids such as, for example, hydrochloric acid, orthophosphoric acid, carboxylic acids such as tartaric acid, citric acid, lactic acid, and sulphonic acids.

According to one new embodiment, the dyeing method comprises applying the composition or ready-to-use composition (produced extemporaneously at the time of use from the coloring and oxidizing compositions described above) to the wet or dry keratin fibers and leaving the composition to act for a waiting time ranging from 1 to 60 minutes, for example, from 10 to 45 minutes, rinsing the fibers and then, optionally, washing the fibers with shampoo, then rinsing them again and drying them.

Specific examples illustrative of the invention are given below, though without being limiting in nature.

### EXAMPLE 1:

The following direct dyeing composition was prepared:

(Expressed in Grams of Active Substance)

| | |
|---|---|
| Direct dye: Basic Blue 99 | 0.1 |
| Silicone: DC2-8299 ® from the company Dow Corning | 2 |
| Ethanol | 20 |
| Hydroxypropylated guar gum: Jaguar HP60 ® sold by Aqualon | 1 |
| C8-C10 alkyl polyglucoside in aqueous solution, comprising 60% | 8 |
| active substance: Oramix CG110 ® sold by SEPPIC | |
| 2-Amino-2-methyl-1-propanol qs pH | 7.5 |
| Demineralized water qs | 100 |

This composition was applied to locks of natural grey hair comprising 90% white hairs for 30 minutes. The hair was subsequently rinsed, washed with a standard shampoo and then dried.

A blue shade was obtained which was highly resistant to several shampooings. Moreover, the cosmetic condition of the fibers was highly satisfactory.

### EXAMPLE 2:

The following oxidation dyeing composition was prepared:

(Expressed in Grams of Active Substance)

| | |
|---|---|
| Paraphenylenediamine | 0.108 |
| 2-Methyl-5-aminophenol | 0.123 |
| Silicone: DC2-8299 ® from the company Dow Corning | 2 |
| Ethanol | 20 |
| C8-C10 alkyl polyglucoside in aqueous solution, comprising | 3.6 |
| 60% active substance: Oramix CG110 ® sold by SEPPIC | |
| Benzyl alcohol | 2 |
| Polyethylene glycol comprising 8 mol of ethylene oxide | 3 |
| Sodium metabisulphite in aqueous solution, comprising 35% | 0.227 |
| active substance | |
| Pentasodium salt of diethylenetriaminepentaacetic acid | 0.48 |
| Aqueous ammonia comprising 20% NH₃ | 6.8 |
| Demineralized water qs | 100 |

This composition was mixed, weight for weight, with 20-volume hydrogen peroxide. The final pH of the mixture was 9.5. The mixture was then applied to grey hair comprising 90% white hairs and was left to act for 30 minutes. The hair was subsequently washed with a standard shampoo and then rinsed with water and dried.

The hair was dyed in a mauvish red shade which was resistant to several shampooings, and the fibers exhibited a highly satisfactory cosmetic condition.

What is claimed is:

1. A composition for dyeing human keratin fibers comprising, in a cosmetically acceptable medium:
(i) at least one coloring agent chosen from direct dyes and oxidation dyes and
(ii) at least one aminosilicone comprising at least one aminoethylimino(C₄-C₈)alkyl group.

2. The composition according to claim 1, wherein the human keratin fibers are hair.

3. The composition according to claim 1, wherein the at least one aminosilicone comprising at least one aminoethylimino(C₄-C₈)alkyl group has the formula (I) below: 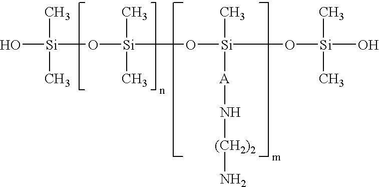
wherein:
A is chosen from linear and branched C₄-C₈ alkylene radicals;
m and n are numbers such that the sum (n+m) ranges from 1 to 2000;
n is a number ranging from 0 to 1999; and
m is a number ranging from 1 to 2000.

4. The composition according to claim 3, wherein the sum (m+n) ranges from 50 to 150.

5. The composition according to claim 3, wherein n is a number ranging from 49 to 149.

6. The composition according to claim 3, wherein m is a number ranging from 1 to 10.

7. The composition according to claim 3, wherein A is chosen from linear and branched C₄ alkylene radicals.

8. The composition according to claim 1, wherein the viscosity of the at least one aminosilicone is greater than 25,000 mm²/s at 25° C.

9. The composition according to claim 8, wherein the viscosity of the at least one aminosilicone ranges from 30,000 to 200,000 mm²/s at 25° C.

10. The composition according to claim 9, wherein the viscosity of the at least one aminosilicone ranges from 30,000 to 150,000 mm²/s at 25° C.

11. The composition according to claim 1, wherein the at least one aminosilicone has a weight-average molecular mass ranging from 2000 to 1,000,000.

12. The composition according to claim 11, wherein the at least one aminosilicone has a weight-average molecular weight ranging from 3500 to 200,000.

13. The composition according to claim 1, wherein the at least one aminosilicone is in the form of an oil-in-water emulsion comprising at least one surfactant.

14. The composition according to claim 13, wherein the oil-in-water emulsion comprises at least one surfactant chosen from cationic and nonionic surfactants.

15. The composition according to claim 1, wherein the particle size of the at least one aminosilicone ranges in size from 3 to 500 nanometers.

16. The composition according to claim 15, wherein the particle size of the at least one aminosilicone ranges from 5 to 300 nanometers.

17. The composition according to claim 16, wherein the particle size of the at least one aminosilicone ranges from 10 to 275 nanometers.

18. The composition according to claim 17, wherein the particle size of the at least one aminosilicone ranges from 150 to 275 nanometers.

19. The composition according to claim 1, wherein the at least one aminosilicone is present in an amount ranging from 0.01 to 20% by weight of the total weight of the composition.

20. The composition according to claim 19, wherein the at least one aminosilicone is present in an amount ranging from 0.1 to 15% by weight relative to the total weight of the composition.

21. The composition according to claim 20, wherein the at least one aminosilicone is present in an amount ranging from 0.5 to 10% by weight relative to the total weight of the composition.

22. The composition according to claim 1, wherein the at least one oxidation dye is chosen from oxidation bases and couplers and their acid addition salts.

23. The composition according to claim 22, further comprising at least one oxidation base wherein said at least one oxidation base is chosen from ortho- and para-phenylenediamines, double bases, ortho- and para-aminophenols, heterocyclic bases, and their acid addition salts.

24. The composition according to claim 23, wherein said para-phenylenediamines are chosen from compounds of formula (II) and their acid addition salts: 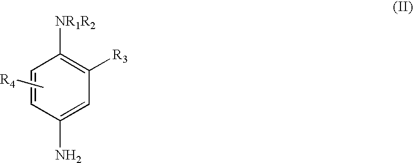
wherein:
R₁ is chosen from hydrogen, C₁-C₄ alkyl groups, C₁-C₄ monohydroxyalkyl groups, C₂-C₄ polyhydroxyalkyl groups, (C₁-C₄)alkoxy(C₁-C₄)alkyl groups, C₁-C₄ alkyl groups substituted by at least one group chosen from nitrogen-containing groups, phenyl groups and 4′-aminophenyl radicals;
R₂ is chosen from hydrogen, C₁-C₄ alkyl groups, C₁-C₄ monohydroxyalkyl groups, C₂-C₄ polyhydroxyalkyl radicals, (C₁-C₄)alkoxy(C₁-C₄)alkyl groups, and C₁-C₄ alkyl groups substituted by at least one group chosen from nitrogen-containing groups;
R₃ is chosen from hydrogen, halogens, C₁-C₄ alkyl groups, sulpho groups, carboxyl groups, C₁-C₄ monohydroxyalkyl groups, C₁-C₄ hydroxyalkoxy groups, C₁-C₄ acetylaminoalkoxy groups, C₁-C₄ mesylaminoalkoxy groups, and C₁-C₄ carbamoylaminoalkoxy radicals, and
R₄ is chosen from hydrogen, halogens, and C₁-C₄ alkyl radicals.

25. The composition according to claim 24, wherein R₁ and R₂ form, with the nitrogen to which they are bonded, a 5- or 6-membered nitrogen-containing heterocycle ring optionally substituted by at least one group chosen from alkyl groups, hydroxyl groups and ureido groups.

26. The composition according to claim 23, wherein the double bases are chosen from compounds of formula (III) and their acid addition salts: 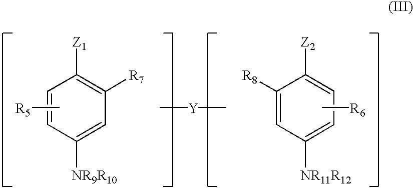
wherein:
Z₁ and Z₂, which may be identical or different, are each chosen from hydroxyl groups and
NH₂ radicals which may be substituted by a group chosen from C₁-C₄ alkyl radicals and a linking arm Y;
the linking arm Y is chosen from linear and branched alkylene groups comprising from 1 to 14 carbon atoms, optionally interrupted by, or optionally terminated with, at least one entity chosen from nitrogen-containing groups and heteroatoms, and optionally substituted with at least one group chosen from hydroxyl groups and C₁-C₆ alkoxy radicals;
R₅ and R₆, which may be identical or different, are each chosen from hydrogen, halogens, C₁-C₄ alkyl groups, C₁-C₄ monohydroxyalkyl groups, C₂-C₄ polyhydroxyalkyl groups, C₁-C₄ aminoalkyl radicals, and a linking arm Y;
R₇, R₈, R₉, R₁₀, R₁₁ and R₁₂, which may be identical or different, are each chosen from hydrogen, a linking arm Y and C₁-C₄ alkyl radicals;
subject to the proviso that the compounds of formula (III) comprise only one linking arm Y per molecule.

27. A composition according to claim 23, wherein said para-aminophenols are chosen from compounds of formula (IV), and their acid addition salts: 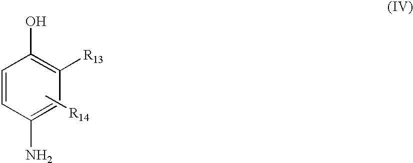
wherein:
R₁₃ is chosen from hydrogen, halogens, C₁-C₄ alkyl groups, C₁-C₄ monohydroxyalkyl groups, (C₁-C₄)alkoxy(C₁-C₄)alkyl groups, C₁-C₄ aminoalkyl groups, and C₁-C₄ hydroxy(C₁-C₄)alkylaminoalkyl radicals and
R₁₄ is chosen from hydrogen, halogens, C₁-C₄ alkyl groups, C₁-C₄ monohydroxyalkyl groups, C₂-C₄ polyhydroxyalkyl groups, C₁-C₄ aminoalkyl groups, C₁-C₄ cyanoalkyl groups, and (C₁-C₄)alkoxy(C₁-C₄)alkyl radicals.

28. The composition according to claim 23, wherein said heterocyclic bases are chosen from pyridine derivatives, pyrimidine derivatives, and pyrazole derivatives.

29. The composition according to claim 23, wherein said at least one oxidation base is present in a concentration ranging from 0.0005 to 12% by weight relative to the total weight of the composition.

30. The composition according to claim 22, further comprising at least one coupler wherein said at least one coupler is chosen from meta-phenylenediamines, meta-aminophenols, meta-diphenols, heterocyclic couplers, and their acid addition salts.

31. The composition according to claim 30, wherein said at least one coupler is present in a concentration ranging from 0.0001 to 10% by weight relative to the total weight of the composition.

32. The composition according to claim 22, wherein said acid-addition salts are chosen from hydrochlorides, hydrobromides, sulphates, tartrates, lactates and acetates.

33. The composition according to claim 1, wherein the direct dyes are chosen from neutral, acidic and cationic nitro benzene direct dyes, neutral, acidic and cationic azo direct dyes, neutral, acidic and cationic quinones, azine direct dyes, triarylmethane direct dyes, indoamine direct dyes, and natural direct dyes.

34. The composition according to claim 33, wherein said direct dyes are anthraquinone direct dyes.

35. The composition according to claim 1, wherein said direct dyes are present in a concentration ranging from 0.001 to 20% by weight relative to the total weight of the composition.

36. The composition according to claim 35, wherein said direct dyes are present in a concentration ranging from 0.005% to 10% by weight relative to the total weight of the composition.

37. The composition according to claim 1, further comprising at least one reducing agent in an amount ranging from 0.05% to 3% by weight relative to the total weight of the composition.

38. The composition according to claim 1, further comprising at least one oxidizing agent.

39. The composition according to claim 38, wherein said at least one oxidizing agent is chosen from hydrogen peroxide, urea peroxide, alkali metal bromates, alkali metal ferricyanides, persalts, and oxidation-reduction enzymes, wherein said oxidation-reduction enzymes are optionally in the presence of their corresponding donor or cofactor.

40. The composition according to claim 39, wherein said at least one oxidizing agent is hydrogen peroxide.

41. The composition according to claim 40, wherein said at least one oxidizing agent is a hydrogen peroxide solution with a titre ranging from 1 to 40 volumes.

42. The composition according to claim 1, wherein said composition has a pH ranging from 4 to 11.

43. A method of dyeing human keratin fibers comprising applying to said human keratin fiber a coloring composition comprising, in a cosmetically acceptable medium,
(i) at least one direct dye, and
(ii) at least one aminosilicone comprising at least one aminoethylimino(C₄-C₈)alkyl group.

44. The method according to claim 43, wherein said human keratin fibers are human hair.

45. A method of dyeing human keratin fibers comprising:
(A) applying to said human keratin fibers a coloring composition comprising, in a cosmetically acceptable medium:
(i) at least one coloring agent chosen from direct dyes and oxidation dyes and
(ii) at least one aminosilicone comprising at least one aminoethylimino(C₄-C₈)alkyl group, and
(B) developing a color, at alkaline, neutral or acidic pH, by applying to said fibers at least one oxidizing composition comprising at least one oxidizing agent.

46. The method according to claim 45, wherein said keratin fibers are human hair.

47. The method according to claim 45, wherein said coloring composition and said oxidizing composition are applied sequentially to the human keratin fiber.

48. The method according to claim 47, wherein said coloring composition and said oxidizing composition are mixed at the time of use.

49. A method of dyeing keratin fibers comprising:
(A) preparing a coloring composition comprising, in a cosmetically acceptable medium:
(i) at least one coloring agent chosen from direct dyes and oxidation dyes and
(ii) at least one aminosilicone comprising at least one aminoethylimino(C₄-C₈)alkyl group,
(B) applying to said fibers the coloring composition,
(C) optionally leaving said coloring composition to act for a waiting time ranging from 1 to 60 minutes.
(D) optionally rinsing said fibers, optionally shampooing said fibers, and further optionally rinsing said fibers, and
(E) optionally drying said fibers.

50. The method according to claim 49, wherein said human keratin fibers are hair.

51. The method according to claim 49, wherein said leaving time is a time ranging from 10 to 45 minutes.

52. The method according to claim 49, wherein said keratin fibers are chosen from wet keratin fibers and dry keratin fibers.

53. A method of dyeing keratin fibers comprising:
(A) applying to said fibers a ready-to-use composition, produced extemporaneously at the time of use from an oxidizing composition and a coloring composition comprising, in a cosmetically acceptable medium:
(i) at least one coloring agent chosen from direct dyes and oxidation dyes and
(ii) at least one aminosilicone comprising at least one aminoethylimino(C₄-C₈)alkyl group,
(B) optionally leaving said ready-to-use composition to act for a waiting time ranging from 1 to 60 minutes,
(C) optionally rinsing said fibers, optionally shampooing said fibers, and further optionally rinsing said fibers, and
(D) optionally drying said fibers.

54. A multi-compartment device or kit for dyeing human keratin fibers comprising multiple compartments, wherein:
(A) a first compartment comprises a composition comprising, in a cosmetically acceptable medium, at least one coloring agent chosen from direct dyes and oxidation dyes and at least one aminosilicone comprising at least one aminoethylimino(C₄-C₈)alkyl group,
(B) a second compartment comprises a composition comprising, in a cosmetically acceptable medium, at least one oxidizing agent.

55. The multi-compartment device or kit according to claim 54, wherein said human keratin fibers are hair.

56. A multi-compartment device or kit for dyeing keratin fibers comprising multiple compartments, wherein:
(A) a first compartment comprises a composition comprising, in a cosmetically acceptable medium, at least one coloring agent chosen from direct dyes and oxidation dyes,
(B) a second compartment comprises a composition comprising, in a cosmetically acceptable medium, at least one oxidizing agent, and
(C) a third compartment comprises a composition comprising, in a cosmetically acceptable medium, at least one aminosilicone comprising at least one aminoethylimino(C₄-C₈)alkyl group.

57. The multi-compartment kit or device according to claim 56, wherein said human keratin fibers are hair.
